(19) United States
(12) Patent Application Publication
(10) Pub. No.: US 2024/0277684 A1
(43) Pub. Date: Aug. 22, 2024

(54) BLOCKADE OF CHEMOKINE (C-C MOTIF) RECEPTOR 2 DURING FLUID RESUSCITATION

(71) Applicant: University of South Florida, Tampa, FL (US)

(72) Inventors: Matthias MAJETSCHAK, Tampa, FL (US); Anthony DESANTIS, Tampa, FL (US); Xianlong GAO, Tampa, FL (US)

(21) Appl. No.: 18/638,133

(22) Filed: Apr. 17, 2024

Related U.S. Application Data
(62) Division of application No. 18/115,058, filed on Feb. 28, 2023.
(60) Provisional application No. 63/268,628, filed on Feb. 28, 2022.

(51) Int. Cl. A61K 31/4439; A61K 31/198; A61P 9/04

(52) U.S. Cl. CPC A61K 31/4439; A61K 31/198; A61P 9/04

(57) ABSTRACT

CCR2 is involved in the regulation of normal cardiovascular function and during the cardiovascular stress response to hemorrhagic shock and fluid resuscitation. Disclosed herein are methods of using CCR2 inhibitors to reduce fluid requirements and to prevent death from hemodynamic decompensation during resuscitation.

### CROSS REFERENCE TO RELATED APPLICATIONS

This application is a divisional of U.S. application Ser. No. 18/115,058, filed Feb. 28, 2023, which claims the benefit of priority to U.S. Provisional Application 63/268,628, filed Feb. 28, 2022, which are incorporated by reference herein in their entireties.

### ACKNOWLEDGEMENT OF GOVERNMENT SUPPORT

This invention was made with government support under Grant No. R01GM139811 awarded by the National Institutes of Health. The government has certain rights in the invention.

### BACKGROUND

Hemorrhagic shock (HS) is the major cause of potentially preventable death after accidental injuries. HS accounts for over 35% of pre-hospital deaths and over 40% of deaths within the first 24 hours in trauma patients (Kauvar D S, et al., Impact of hemorrhage on trauma outcome: an overview of epidemiology, clinical presentations, and therapeutic considerations. J Trauma 2006; 60(6 Suppl):S3-111). Despite the urgent need to improve outcomes from HS, treatment options are limited. Pharmacological approaches to improve fluid resuscitation and reduce adverse effects associated with current treatment strategies, such as fluid overload, are not available. Such drugs, however, would likely have a significant clinical impact and could save the lives of many patients (Id., Heron M, et al., Deaths: final data for 2006. Natl Vital Stat Rep 2009; 57(14):1-134).

It is known that tissue injury and hepatic hypoxia during traumatic-hemorrhagic shock (T/HS) drive inflammation, which contributes to vascular dysfunction, impaired endothelial barrier function and hemodynamic instability. Chemokines, such as chemokine (C-C motif) ligand 2 (CCL2) or CCL5 have been identified as key drivers that initiate and amplify the very early inflammatory response to T/HS and fluid resuscitation in animals and humans (Ziraldo C, et al., Central role for MCP-1/CCL2 in injury induced inflammation revealed by in vitro, in silico, and clinical studies. PLoS One 2013; 8(12):e79804; Almahmoud K, et al. Impact of Injury Severity on Dynamic Inflammation Networks Following Blunt Trauma. Shock 2015; 44(2):101-109; Hsieh C H, et al., The role of MIP-1 alpha in the development of systemic inflammatory response and organ injury following trauma hemorrhage. J Immunol 2008; 181(4):2806-2812; Makley A T, et al., Resuscitation with fresh whole blood ameliorates the inflammatory response after hemorrhagic shock. J Trauma 2010; 68(2):305-311; Richter J R, et al., Macrophage-derived chemokine (CCL22) is a novel mediator of lung inflammation following hemorrhage and resuscitation. Shock 2014; 42(6):525-531). Systemic pre-hospital CCL2 concentrations have been shown to be significantly increased in hypotensive trauma patients, as compared with normotensive trauma patients, and systemic CCL2 concentrations within 24 hours of admission have been reported to segregate surviving from non-surviving trauma patients (Ziraldo C, Id.; Almahmoud K, et al., Prehospital Hypotension Is Associated With Altered Inflammation Dynamics and Worse Outcomes Following Blunt Trauma in Humans. Crit Care Med 2015; 43(7):1395-1404). Although the mechanisms underlying these important clinical correlations are poorly understood, they suggest that CCL2 release may contribute to blood pressure regulation and the development of hemodynamic instability during the early inflammatory response to T/HS. CCL2 is the principal endogenous agonist of chemokine (C-C motif) receptor 2 (CCR2)(Alexander S P, et al., THE CONCISE GUIDE TO PHARMACOLOGY 2017/18: G protein-coupled receptors. Br J Pharmacol 2017; 174 Suppl 1:S17-S129). CCR2 is expressed on various immune cells, vascular endothelial and smooth muscle cells, and in multiple other organs and tissues. While CCL2 is also an agonist at CCR3 and CCR5, the binding affinity of CCL2 for these receptors is more than 100-fold lower than the binding affinity of CCL2 for CCR2 (Kd»0.5 nM) (Napier C, et al., Molecular cloning and radioligand binding characterization of the chemokine receptor CCR5 from rhesus macaque and human. Biochem Pharmacol 2005; 71(1-2):163-172; Daugherty B L, et al., Cloning, expression, and characterization of the human eosinophil eotaxin receptor. J Exp Med 1996; 183(5):2349-2354; Coulin F, et al., Characterisation of macrophage inflammatory protein-5/human CC cytokine-2, a member of the macrophage inflammatory-protein family of chemokines. Eur J Biochem 1997; 248(2):507-515).

Due to the important roles of chemokine receptors in numerous disease processes, various selective chemokine receptor antagonists have been developed, among which the CCR5 antagonist Maraviroc and the CXCR4 antagonist AMD3100 are approved by the Federal Drug Adminstration (Xue C B, et al., Discovery of INCB3284, a Potent, Selective, and Orally Bioavailable hCCR2 Antagonist. ACS Med Chem Lett 2011; 2(6):450-454; White J R, et al., Identification of potent, selective non-peptide CC chemokine receptor-3 antagonist that inhibits eotaxin-, eotaxin-2-, and monocyte chemotactic protein-4-induced eosinophil migration. J Biol Chem 2000; 275(47):36626-36631; Lai W Y, et al., Latest update on chemokine receptors as therapeutic targets. Biochem Soc Trans 2021; 49(3):1385-1395; Woollard S M, et al., Maraviroc: a review of its use in HIV infection and beyond. Drug Des Devel Ther 2015; 9:5447-5468; De Clercq E: Mozobil® (Plerixafor, AMD3100), 10 years after its approval by the US Food and Drug Administration. Antivir Chem Chemother 2019; 27:2040206619829382).

INCB3284 is a selective CCR2 antagonist which reached phase II trials in patients with rheumatoid arthritis (Xue C B, Id.; Mackay C R: Moving targets: cell migration inhibitors as new anti-inflammatory therapies. Nat Immunol 2008; 9(9):988-998). The effects of INCB3284 during resuscitation from HS, however, have not been evaluated. The present disclosure demonstrates that blocking the major CCL2 receptor CCR2 with the bona-fide antagonist INCB3284 affect hemodynamics and fluid requirements in HS and fluid resuscitation models in rats.

### SUMMARY

In one aspect, disclosed herein is a method of fluid resuscitation of a patient in need thereof comprising administering to the patient a resuscitation fluid and an effective amount of a C-C chemokine receptor (CCR) inhibitor. In a further aspect, disclosed herein is a resuscitation fluid comprising a CCR inhibitor and an aqueous iso or hypertonic salt solution.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects described below.

FIGS. 1A-1C: Effects of INCB3284 on intrinsic vascular function and on blood pressure in normal rats. FIG. 1A. Pressure myography with isolated rat mesenteric arteries. Arteries were exposed to vehicle or 10 mM of INCB3284 for 15 min, followed by increasing doses of phenylephrine (PE). Constriction (% o.d.): constriction in percent of the outer diameter of the artery at baseline before exposure to vehicle or INCB3284. Data are mean±SE, n=4 from 4 different animals per condition. FIGS. 1B and 1C. Animals received repetitive injections of 1 mL vehicle (Lactated Ringers solution, LR, FIG. 1B) or increasing doses of INCB3284 in 1 mL vehicle (FIG. 1C). SBP: systolic blood pressure. DBP: diastolic blood pressure. MAP: mean arterial blood pressure. Blood pressures are provided in mmHg. Arrows indicate time points of vehicle or drug injection. Data are mean±SE, n=3/group.

FIGS. 2A-2F: Fluid requirements to maintain hemodynamics after hemorrhagic shock. All data are mean±SE. Arrows: time points of drug injection. FIGS. 1A and 1B. Animals were treated with vehicle. FIGS. 1C and 1D. Animals were treated with 1.1 mmol/kg (light grey squares, n=4) and 5.5 mmol/kg INCB3284 (dark grey squares, n=5). FIGS. 1E and 1F. Animals were treated with 5.5 mmol/kg Maraviroc (n=3). FIGS. 1A, 1C, and 1E. MAP (mmHg). B/D/F. Fluid requirements (mL/kg) to achieve MAP of 60 mmHg or SBP of 90 mmHg. *: p<0.05 vs. vehicle treated animals.

FIGS. 3A-3G: INCB3284 reduces fluid requirements and prevents hemodynamic decompensation during resuscitation from hemorrhagic shock. All data are mean±SE. Arrows: time points of vehicle/drug injection. FIGS. 3A-3C, 3F, and 3G. Open circles: vehicle treated animals (n=10). Dark squares: INCB3284 (5 mmol/kg) treated animals (n=8). *: p<0.05 vs. vehicle treated animals. FIG. 3A. Hemorrhage volumes to achieve mean arterial blood pressure (MAP) of 30 mmHg. % TBV: percent total blood volume. FIG. 3B. MAP (mmHg). FIG. 3C. Fluid requirements to achieve MAP of 60 mmHg or SBP of 90 mmHg. FIG. 3D. Fluid requirements to achieve MAP of 60 mmHg or SBP of 90 mmHg of all individual vehicle-treated animals. Short arrows indicate the time point when fluid resuscitation requirements to maintain target blood pressures increased, which was defined as beginning of hemodynamic decompensation. FIG. 3E. Fluid requirements to achieve MAP of 60 mmHg or SBP of 90 mmHg of all individual INCB3284-treated animals. FIG. 3F. Time to the beginning of hemodynamic decompensation. FIG. 3G. Kaplan Meier survival curve.

FIGS. 4A-4D: INCB3284 reduces tissue wet/dry weight ratios after resuscitation from hemorrhagic shock. Wet-weight/dry-weight ratios of lung (FIG. 4A), small bowel (FIG. 4B), colon (FIG. 4C) and all tissues combined (FIG. 4D). Boxes extend from the 25th to 75th percentile, the horizontal line shows the median. Error bars show the range of data (minimum/maximum). The level of statistical significance is indicated.

FIGS. 5A-5C. Single dose SB328437 treatment reduces fluid resuscitation requirements after 30 min of hemorrhagic shock. All data are mean±SE. Arrows represent the time point of drug/vehicle injection. Open circles: animals treated with vehicle (n=5). Open triangles: animals treated with 0.25 μmol/kg SB328437 (n=3). Grey triangles: animals treated with 1.1 μmol/kg SB328437 (n=3). *: p<0.05 vs. animals treated with vehicle. (FIG. 5A) Hemorrhage volumes for maintain mean arterial blood pressure (MAP of 30 mmHg). % TBV: percent of total blood volume. (FIG. 5B) MAP (mmHg). (FIG. 5C) Fluid resuscitation in mL/kg required to maintain MAP of 60 mmHg or systolic blood pressure. (SBP) of 90 mmHg.

FIGS. 6A-6H. Single dose INCB3284 treatment transiently reduces fluid resuscitation requirements after 60 min of hemorrhagic shock. All data are mean±SE. Arrows represent the time point of drug/vehicle injection. Open circles: animals treated with vehicle (n=6). Grey triangles: animals treated with 1.1 μmol/kg SB328437 (n=6). Grey squares: animals treated with 5 μmol/kg INCB3284 (n=6). *: p<0.05 vs. animals treated with vehicle. (FIG. 6A) Hemorrhage volumes for maintain mean arterial blood pressure (MAP of 30 mmHg). % TBV: percent of total blood volume. (FIG. 6B) MAP (mmHg). (FIG. 6C) Fluid resuscitation in mL/kg required to maintain MAP of 60 mmHg or systolic blood pressure (SBP) of 90 mmHg. (FIG. 6D) Hct %: Hematocrit values in %. (FIG. 6E) PaO₂: Partial pressure of oxygen in arterial blood. (FIG. 6F) PaCO₂: Partial pressure of carbon dioxide in arterial blood. (FIG. 6G) Plasma lactate concentration (mmol/L). (FIG. 6H) Kaplan-Meier survival curve.

FIGS. 7A-7H. Redosing of INCB3284 reduces fluid resuscitation requirements after 60 min of hemorrhagic shock. All data are mean±SE. Arrows represent the time points of drug/vehicle injection. Open circles: animals treated with vehicle (n=7). Grey squares: animals treated with 5 μmol/kg INCB3284 (n=7). *: p<0.05 vs. animals treated with vehicle. (FIG. 7A) Hemorrhage volumes for maintain mean arterial blood pressure (MAP of 30 mmHg). % TBV: percent of total blood volume. (FIG. 7B) MAP (mmHg). (FIG. 7C) Fluid resuscitation in mL/kg required to maintain MAP of 60 mmHg or systolic blood pressure (SBP) of 90 mmHg. (FIG. 7D) Hct %: Hematocrit values in %. (FIG. 7E) PaO₂: Partial pressure of oxygen in arterial blood. (FIG. 7F) PaCO₂: Partial pressure of carbon dioxide in arterial blood. (FIG. 7G) Plasma lactate concentration (mmol/L). (FIG. 7H) Kaplan-Meier survival curve.

FIGS. 8A-8B. SB328437 and INCB3284 do not affect survival during lethal hemorrhage without fluid resuscitation. All data are mean±SE. Arrows represent the time point of drug/vehicle injection. Open circles: animals treated with vehicle (n=3). Grey triangles: animals treated with 1.1 μmol/kg SB328437 (n=5). Grey squares: animals treated with 5 μmol/kg INCB3284 (n=5). (FIG. 8A) MAP (mmHg). (FIG. 8B) Kaplan-Meier survival curve.

### DETAILED DESCRIPTION

The materials, compounds, compositions, and methods described herein may be understood more readily by reference to the following detailed description of specific aspects of the disclosed subject matter, and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below. Additional definitions are set forth throughout this disclosure.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

As used herein, the term “about” or “approximately” refers to a quantity, level, value, number, frequency, percentage, dimension, size, amount, weight or length that varies by acceptable levels in the art. In some embodiments, the amount of variation may be as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

A numerical range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, an “effective amount” means the dose to be administered to a subject and the frequency of administration to the subject which is readily determined by one of ordinary skill in the art, by the use of known techniques and by observing results obtained under analogous circumstances. In determining the effective amount or dose, a number of factors are considered by the attending diagnostician, including, but not limited to, the potency and duration of action of the compounds used; the nature and severity of the condition to be treated as well as on the sex, age, weight, general health, and individual responsiveness of the patient to be treated, and other relevant circumstances.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

By “reduce” or other forms of the word, such as “reducing” or “reduction,” is meant lowering of an event or characteristic (e.g., inflammation). It is understood that this is typically in relation to some standard or expected value, in other words it is relative, but that it is not always necessary for the standard or relative value to be referred to. For example, “reduces inflammation” means reducing the rate of growth of a tumor relative to a standard or a control.

As used herein, by a “patient” is meant an individual or subject under the treatment of a clinician, e.g., physician. Thus, the “patient” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Patient” can also include a mammal, such as a primate or a human. Thus, the patient can be a human or veterinary patient.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

The term “therapeutically effective” refers to the amount of the composition used is of sufficient quantity to ameliorate one or more causes or symptoms of a disease or disorde. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Reference will now be made in detail to specific aspects of the disclosed materials, compounds, compositions, articles, and methods, examples of which are illustrated in the accompanying Examples and Figures.

### Methods

The standard of care in the initial management of shock includes rapid administration of large volumes of isotonic crystalloid solution, which can be up to several liters in an adult patient. In situations where fluid addition to the vascular system is required, such as for resuscitation, the practice has been to add isotonic fluids in sufficient quantity to replenish vascular fluid volume. In practice, this has often been at a rate of 1:1 as compared to blood loss, and often as high as 3:1 compared to blood loss due to physiologic equilibration of resuscitative fluid between the intravascular and interstitial space. Advantages of this practice were avoidance or reduction in triggering anti-inflammatory response, the provision of oxygen to the cells, and the replenishment of osmotic pressure in the vascular system. On the other hand, large volumes of fluids are required to be administered, and cell death often occurs despite the additions of large volumes of the fluids due to cells lapsing into a regime of anaerobic metabolism from which they could not recover. Thus, disclosed herein are methods of administering resuscitation fluids to patients in a manner that can reduce the volume of fluid needed.

Disclosed herein, in one aspect, are methods of fluid resuscitation of a patient in need thereof comprising administering to the patient a resuscitation fluid and an effective amount of a C-C chemokine receptor (CCR) inhibitor.

Also disclosed are methods of reducing the resuscitation fluid requirements for fluid resuscitation by administering to a patient in need of fluid resuscitation a CCR inhibitor. Such patients can be hypotensive and/or hemodynamically instable patients. Also disclosed are methods of preventing or delaying hemodynamic decompensation in patients with shock comprising administering to a patient in need thereof a resuscitation fluid and a CCR inhibitor.

Still further, disclosed are methods for preventing or treating septic shock, hemorrhagic shock, hypotension, acidosis, and/or hypovolemia comprising administering to a patient in need of treatment or prevention thereof a resuscitation fluid and a CCR antagonist.

Further, suitable patients that can be treated with the disclosed methods can be patients having hemorrhagic shock, e.g., traumatic-hemorrhagic shock. Alternatively, suitable patients can be those undergoing cardiovascular, abdominal, or transplant surgery, e.g., a percutaneous coronary intervention, a cardiac bypass surgery, a fibrinolytic therapy, a stent placement. Patients being treated for ischemia, hypovolemic shock, myocardial infarction, or stroke are also suitable patients for the disclosed methods. In further examples, the patients can be treated for trauma, burn, sepsis, or shock.

The CCR inhibitor can be INCB3284 or SB328437. The CCR inhibitor can be administered in an effective amount, which for example can be from 1 to 10 mmol/kg of the patient, e.g., from 1 to 5, from 5 to 10, from 3 to 7, from 2 to 4, from 6 to 8, or from 4 to 6 mmol/kg.

In the disclosed methods, the CCR inhibitor can be administered to the patient separately from the resuscitation fluid. That is, the CCR inhibitor can be administered in a separate composition from the resuscitation fluid. The CCR inhibitor can be administered before, concurrently, or after administering the resuscitation fluid. In other examples, the CCR inhibitor can be a component of the resuscitation fluid.

The CCR inhibitor can be administered in one or two doses, preferably in one dose. Still in other examples the CCR inhibitor can be administered in more than two doses or continuously as a component of the resuscitation fluid.

The resuscitation fluid can be Ringer's solution or lactated Ringer's solution. In general, any resuscitation fluid can be used. For example, in some cases the resuscitation fluid can comprise whole blood.

Resuscitation fluids have typically been used a dosage rate of about 3 times the amount of blood loss. Stated differently, for every liter of blood loss, a resuscitation fluid treatment required 3 liters of resuscitation fluid. Recommended continued treatment is based on the observed response to the initial fluid therapy. American College of Surgeons, 154, 585-588, (1987). As a general rule, guidelines are based on the “three for one” rule. This is based on the long-standing empirical observation that most hemorrhagic shock patients require up to 300 mL of electrolyte solution for each 100 mL of blood lost. The present methods, however, are intended to use resuscitation fluids in dosages less than the total amount of blood loss. Thus, disclosed herein are methods whereby the resuscitation fluid is administered in an amount that is less than three times the amount of fluid lost by the patient, e.g., 2.5 times, 2 times, 1.5 times, the same, or less than the amount of fluid (e.g., blood), lost by the patient.

The resuscitation fluid and/or CCR inhibitor can be administered to the patent by intraperitoneal injection, intravenous administration, subcutaneous administration, sublingual administration, inhalation, oral administration, topical application to a blood vessel, or coating of a device to be placed within the subject.

In further examples, the patient can be administered the resuscitation fluid for up to 4 hours. In other examples, the patient can be administered the resuscitation fluid for great than 4 hours, e.g., from 4 to 12 hours.

The disclosed methods can further comprise the additional administration of other active compounds. These additional active compounds include but are not limited to antibiotics, analgesics, anti-inflammatory drugs, antihistamines, sedatives, corticosteroids, electrolytes, gastro-intestinal drugs, muscle relaxants, nutritional agents, vitamins, stimulants, and antiviral agents.

### Compositions

Disclosed herein is a resuscitation fluid comprising a CCR inhibitor and an aqueous iso or hypertonic salt solution. A preferred solution is Ringer's solution or lactated Ringer's solution, although normal saline or other similar isotonic crystalloid solutions can be used. For example, isotonic fluid replacement solutions where isotonic crystalloid solutions are mixed with macromolecular solutions of plasma proteins or synthesized molecules with similar oncotic properties (colloids) including albumin, dextran, hetastarch or polygelatin in NaCl.

In the disclosed compositions, there is added a CCR inhibitor. For example, the CCR inhibitor can be INCB3284 or SB328437.

INCB3284 is selective and orally bioavailable human CCR2 antagonist, inhibiting monocyte chemoattractant protein-1 binding to hCCR2, with an IC₅₀ of 3.7 nM. INCB3284 is commercially available and has the following structure.

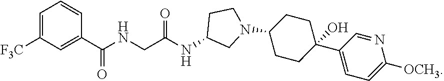 [SMILES: COc1ccc([C@]2(O)CC[C@@H](N3CC[C@@H](NC(=O)CNC(=O)c4cccc(C(F)(F)F)c4)C3)CC2)cn1]

SB328437 is a selective non-peptide CCR3 antagonist with an IC₅₀ of 4.5 nM. It is commercially available and has the following structure.

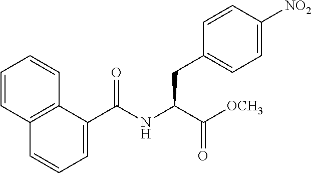 [SMILES: COC(=O)[C@H](Cc1ccc([N+](=O)[O-])cc1)NC(=O)c1cccc2ccccc12]

The ionic salt of the present fluid is preferably a pharmaceutically acceptable salt that ionizes to provide osmotic pressure in an aqueous solution. Preferred salts include sodium chloride (NaCl) and potassium chloride (KCl). NaCl is an especially preferred salt for use as the agent to augment intravascular fluid. In preferred embodiments, the salt is in the form of a saline solution. Those having ordinary skill in the art will recognize that saline is a solution of NaCl in sterile water, used commonly for intravenous infusion.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

The disclosed resuscitation fluid can also be used as a storage solution for organs during organ transplant, for wound irrigation, as a solution for urological and gynecological procedures, to treat intracranial hypertension from head injury, and to help reduce contrast induced nephrotoxicity. Pre-hospital uses for the present resuscitation fluid include ambulance use, battlefield use, emergency room use, trauma, and intensive care use. Similarly, the present resuscitation fluid may be utilized for veterinary use in the same manner it is utilized from human use.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

INCB3284 and Maraviroc were purchased from Tocris (Minneapolis, MN, USA). Phenylephrine was obtained from Millipore Sigma (St. Louis, MO, USA).

### Example 1

In vivo animal experiments. All procedures were performed in accordance with the National Institutes of Health Guidelines for Use of Laboratory Animals and were approved by the University of South Florida Institutional Animal Care and Use Committeee (IS00008139). Male Sprague-Dawley rats (300-400 g, Envigo, Indianapolis, IN, USA). were anesthetized with 1.7% isoflurane via nose-cone inhalation with the SomnoSuite anesthesia system (Kent Scientific, Torrington, CT, USA). At this dose rats did not respond to noxious stimuli but maintained spontaneous respiration. The left femoral artery was isolated and cannulated with a 24-gauge peripheral intravenous catheter to allow for blood withdrawal, hemodynamic monitoring, drug administration, and fluid resuscitation. Hemodynamics were continuously monitored with the SurgivetV6400 blood pressure monitor (Med-Electronics, Beltsville, MD, USA), and blood pressures and heart rate were recorded at 1-10 min intervals. All animals were observed over a 10 min period to ensure hemodynamic stability before start of the experiments. All experiments were performed randomized in alternating order.

To test the effects of INCB3284 in normal rats, animals received repetitive injections of 1 mL of Lactated Ringer's solution (LR, vehicle) in 10 min intervals (n=3) or increasing doses of the drug (0.55, 0.82, 1.4 and 2.75 μmol/kg INCB3284) in 1 mL LR in 10 min intervals (n=3).

To test the effects of INCB3284 and Maraviroc after HS, a Wiggers fixed pressure hemorrhage and fluid resuscitation model was used as described (Babu F S, et al., Chemokine (C-X-C motif) receptor 4 regulates lung endothelial barrier permeability during resuscitation from hemorrhagic shock. Physiol Res 2019; 68(4):675-679; Nassoiy S P, et al., Effects of the Kv7 voltage-activated potassium channel inhibitor linopirdine in rat models of haemorrhagic shock. Clin Exp Pharmacol Physiol 2018; Nassoiy S P, et al., Kv7 voltage-activated potassium channel inhibitors reduce fluid resuscitation requirements after hemorrhagic shock in rats. J Biomed Sci 2017; 24(1):8; Bach H Ht, et al., Proteasome inhibition prolongs survival during lethal hemorrhagic shock in rats. J Trauma Acute Care Surg 2013; 74(2):499-507; Bach H Ht, et al., Chemokine (C-X-C motif) receptor 4 and atypical chemokine receptor 3 regulate vascular alpha(1)-adrenergic receptor function. Mol Med 2014; 20:435-447), with slight modifications. In brief, rats were hemorrhaged to a mean arterial blood pressure (MAP) of less than or equal to 30 mmHg for a period of 30 minutes. At the end of the HS period (t=30 min), animals were injected with either vehicle (1 mL LR) or drug in 1 mL LR, followed by fluid resuscitation with 1 mL bolus injections of LR to maintain a systolic blood pressure (SBP) of 90 mmHg or a MAP of 60 mmHg. To avoid fluid overload, bolus injections of LR were limited to 1 mL/min. In the first series of experiments, animals received 1 mL of LR (n=9), 1.1 μmol/kg (n=4) or 5.5 μmol/kg (n=5) INCB3284 in 1 mL LR or 5.5 mmol/kg Maraviroc (n=3) in 1 mL LR at t=30 min. Animals were then resuscitated until t=90 min. At t=0, 15, 30, 60 and 90 min, blood samples were obtained, and plasma prepared. Samples were stored at −70° C. until further processing. At t=90 min, animals were euthanized (isoflurane inhalation, bilateral pneumothorax).

In the second series of experiments, animals received 1 mL of LR (n=10) or 5 μmol/kg INCB3284 in 1 mL LR (n=8) at t=30 min. Animals were resuscitated until t=300 min. Blood samples were obtained for arterial blood gas analyses and measurements of routine laboratory parameters at time points t=0, 30, 90, 150, 210, 270 and 300 min. Death was defined as asystole or loss of a pulse pressure. At the end of the experiments, surviving animals were euthanized as described before. In animals that survived until t=300 min, a gross necropsy was performed after euthanasia, and tissue from lung, small intestine, and colon was collected for measurements of wet-weight/dry-weight ratios.

Pressure myography. Pressure myography with rat mesenteric arteries was performed as described in detail previously (Albee L J, et al., alpha1-Adrenergic Receptors Function Within Hetero-Oligomeric Complexes With Atypical Chemokine Receptor 3 and Chemokine (CX-C motif) Receptor 4 in Vascular Smooth Muscle Cells. J Am Heart Assoc 2017; 6(8):e006575; Tripathi A, et al., Heteromerization of chemokine (C-X-C motif) receptor 4 with alpha1A/B-adrenergic receptors controls alpha1-adrenergic receptor function. Proc Natl Acad Sci USA 2015; 112(13):E1659-1668; Albee L J, et al., Identification and functional characterization of arginine vasopressin receptor 1A: atypical chemokine receptor 3 heteromers in vascular smooth muscle. Open Biol 2018; 8(1):170207; Bach H H, et al., Chemokine (C-X-C motif) receptor 4 and atypical chemokine receptor 3 regulate vascular alpha(1)-adrenergic receptor function. Mol Med 2014; 20:435-447; DeSantis A J, et al., Chemokine receptor antagonists with alpha1-adrenergic receptor blocker activity. J Basic Clin Physiol Pharmacol 2021). In brief, after euthanasia, the mesentery was removed, third- or fourth-order mesenteric arteries were dissected free from the mesentery, mounted onto two glass cannulae and pressurized to 60 mmHg in a DMT110P pressure myograph (DMT-USA). The vessel bath solution was continuously aerated with 95% O₂, 5% CO₂ throughout the experiment. The outer diameter (o.d.) of the pressurized vessel was then continuously measured and recorded via digital videoedge detection. INCB3284 (10 μM) or vehicle were added to the vessel bath. After 15 min, increasing doses of phenylephrine were added in 10 min intervals.

Arterial blood gases and routine laboratory parameters. Arterial blood gases, electrolytes, creatinine, lactate, hematocrit and hemoglobin were analyzed using the Element point of care veterinary blood gas, electrolyte and critical care analyzer (Cuattro Veterinary USA, Loveland, CO, USA).

Protein measurements. Total protein concentrations in plasma were determined on a Nanodrop 1000 (Thermo Scientific, Ashville, NC). Bovine serum albumin served as protein standard. Due to variable degrees of hemolysis in the recovered plasma, sample absorption at 1=413 nm was measured, the hemoglobin concentration in the sample calculated according to (Pluim D, et al., Correction of peripheral blood mononuclear cell cytosolic protein for hemoglobin contamination. Anal Bioanal Chem 2013; 405(7):2391-2395) and subtracted from the total protein concentration.

Measurements of chemokine concentrations. Chemokine concentrations in plasma were measured with commercially available enzyme linked immunosorbent assays (ELISA) according to the manufacturers' instructions. ELISA kits for CCL2 and CCL5 were purchased from R&D systems (Minneapolis, MN, USA) and for CCL7, CCL11 and CXCL10 from MyBioSource (San Diego, CA, USA). Chemokine concentrations were expressed per mg of total protein corrected for hemoglobin to account for dilutional effects due to continuous fluid resuscitation.

Wet-weight/dry-weight ratios. The ratio of the tissue wet weight to dry weight was determined gravimetrically, as previously described (Nassoiy S P, et al., Pharmacological modulation of C-X-C motif chemokine receptor 4 influences development of acute respiratory distress syndrome after lung ischaemia-reperfusion injury. Clin Exp Pharmacol Physiol 2018; 45(1):16-26; Garcia-Covarrubias L, et al., Ubiquitin enhances the Th2 cytokine response and attenuates ischemia-reperfusion injury in the lung. Crit Care Med 2008; 36(3):979-982; Geng Q, Romero J, Saini V, et al: A subset of 26S proteasomes is activated at critically low ATP concentrations and contributes to myocardial injury during cold ischemia. Biochem Biophys Res Commun 2009; 390(4):1136-1141; Baker T A, et al., Effects of exogenous ubiquitin in a polytrauma model with blunt chest trauma. Crit Care Med 2012; 40(8):2376-2384).

Data analyses and statistics. Data are presented as mean±standard error (SE) or Median with interquartile range (25th/75th percentile). Data were analyzed by Student's t-test, 1-way analysis of variance (ANOVA) or 2-way ANOVA with Dunnett's multiple comparisons tests, as appropriate. Survival curves were analyzed using the log-rank test. Proportions were compared with the Fisher's exact test. Blood pressure trends and dose responses were analyzed with linear and non-linear regression analyses, respectively. All data analyses were calculated with the GraphPad Prism program, Version 9.3.1. A two-tailed p<0.05 was considered significant.

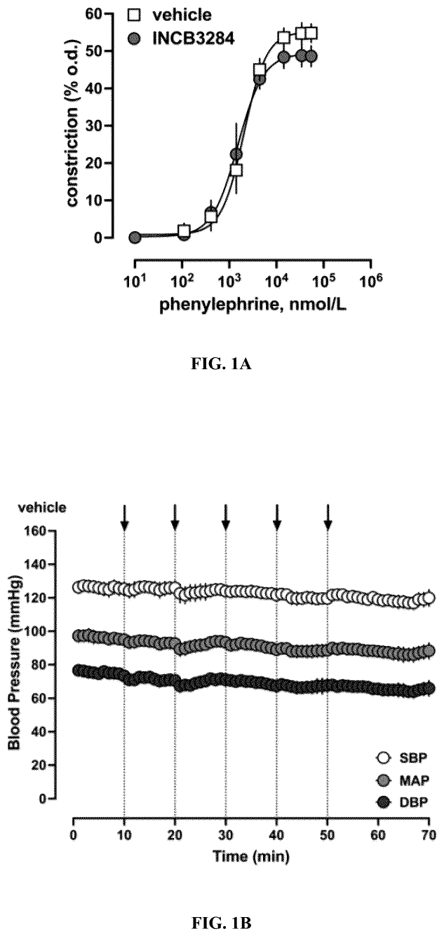

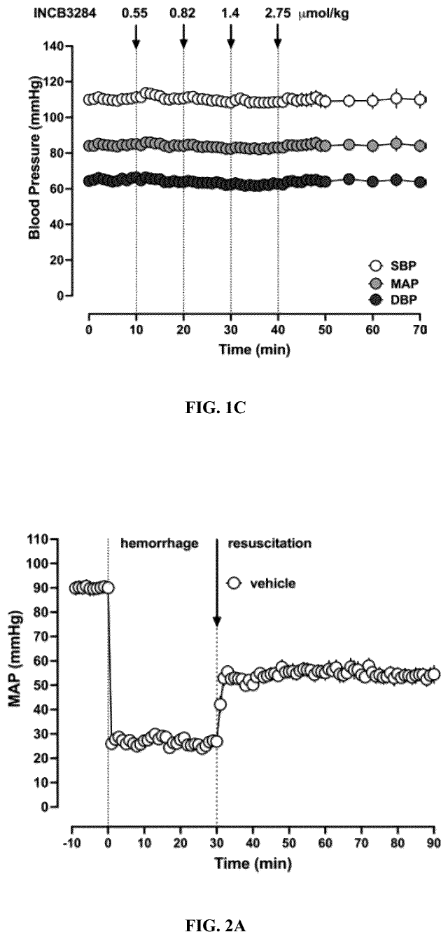

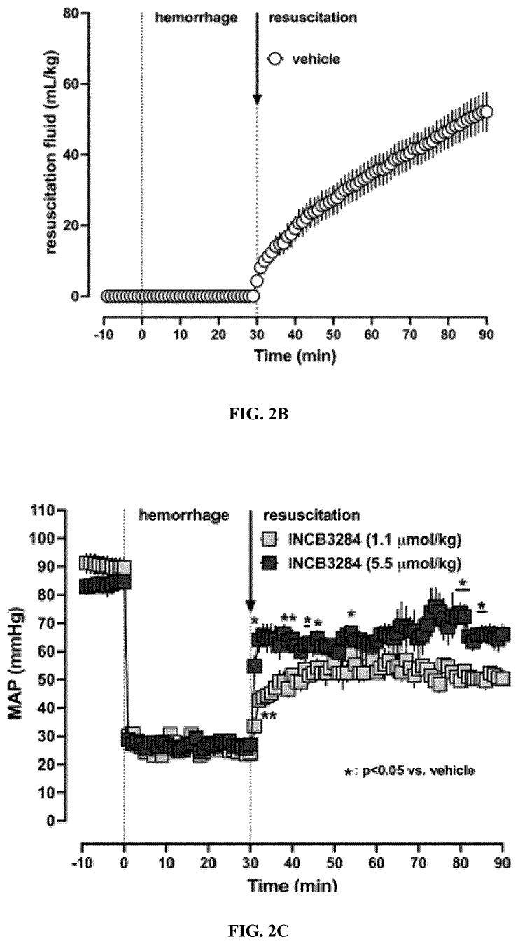

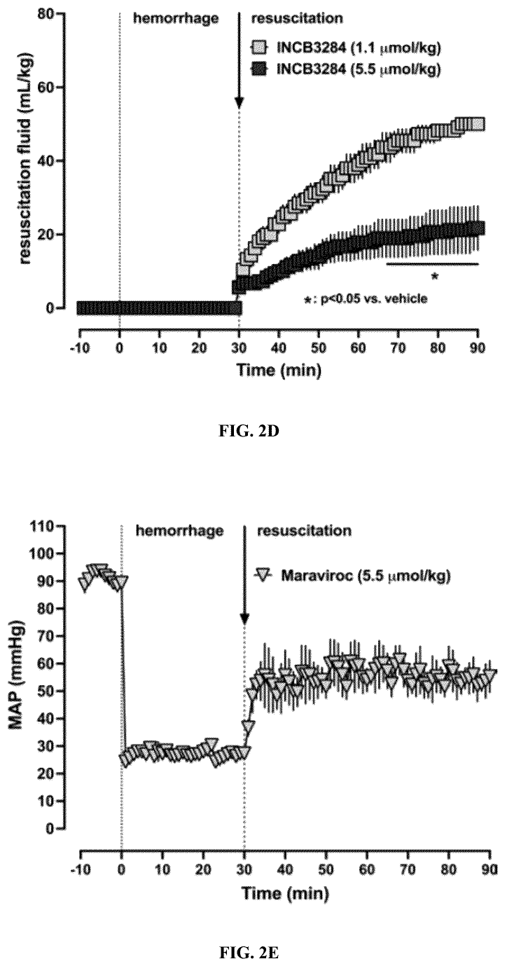

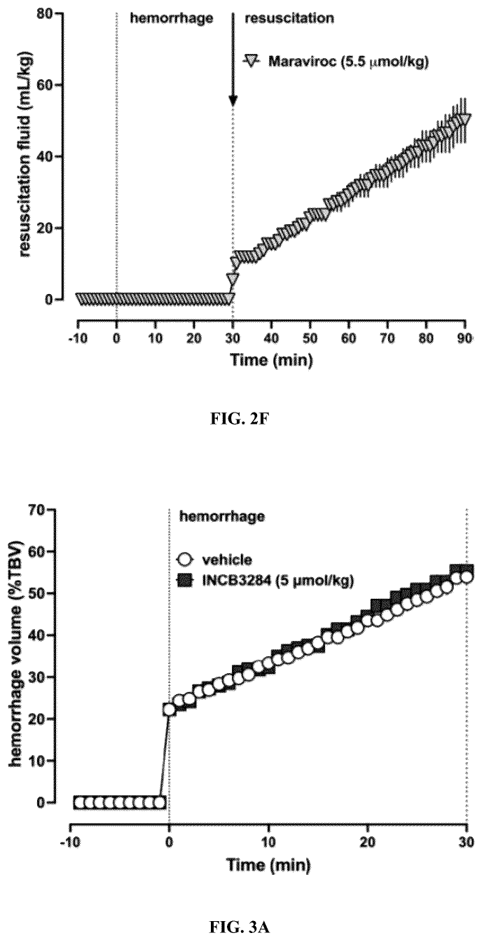

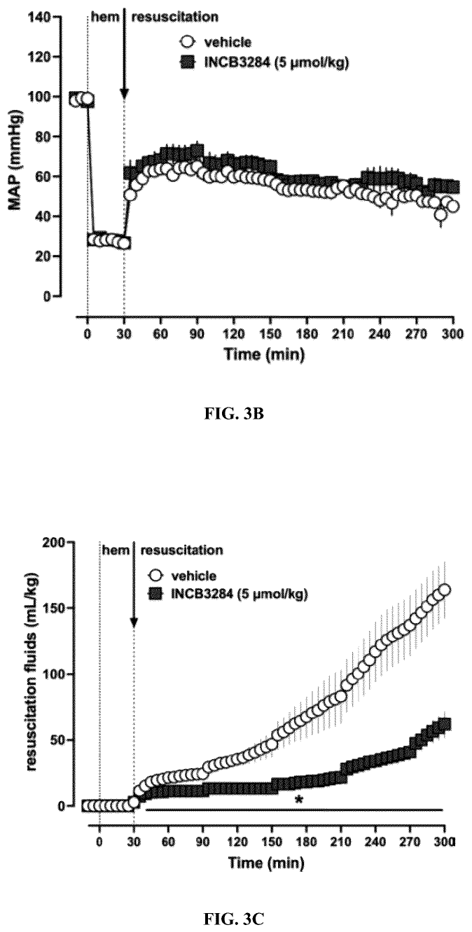

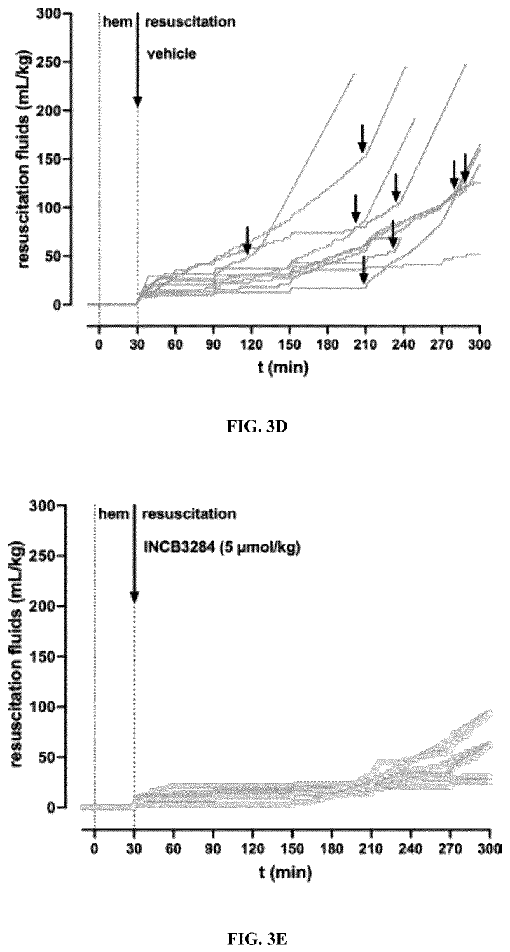

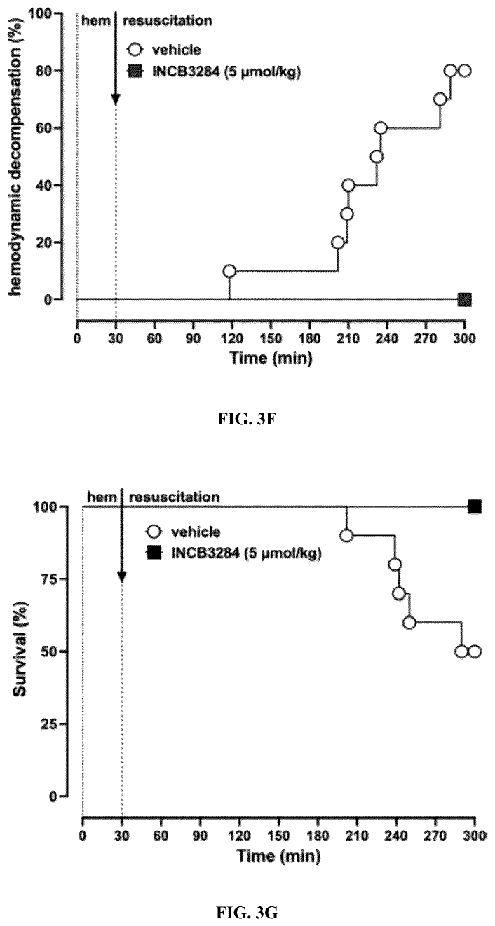

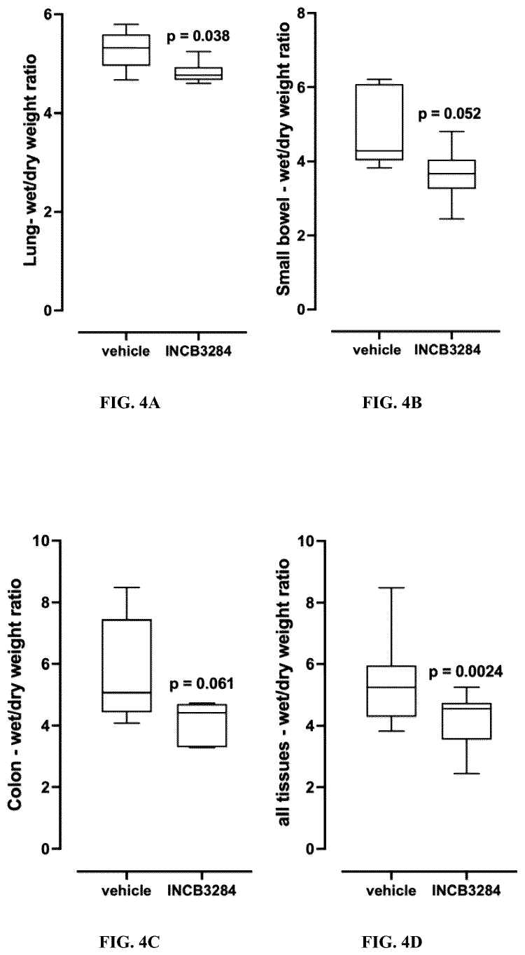

Pressure myography was employed to exclude that INCB3284 would affect function of isolated rat resistance arteries and vasoconstriction induced by the selective al-adrenoceptor agonist phenylephrine (FIG. 1A). As compared with arteries pre-exposed to vehicle, pre-exposure of arteries to 10 μM INCB3284 did not alter artery diameter or affect phenylephrine-induced vasoconstriction. Moreover, whether injection of INCB3284 would affect blood pressure in normal rats was tested. As shown in FIG. 1B, bolus injections of 1 mL of vehicle had no acute blood pressure increasing effects. Linear regression analysis showed that MAP in vehicle-treated animals continuously decreased by 0.09±0.01 mmHg/min (95% confidence interval: 0.1-0.07 mmHg/min; r2: 0.25), which is significantly non-zero (p<0.001). While injection of increasing doses of INCB3284 in a total volume of 1 mL also did not show acute effects on blood pressure, MAP remained constant during the observation period (FIG. 1C). The slope of the MAP linear regression line was −0.011 mmHg/min (95% confidence interval:−0.04-0.016 mmHg/min; r2: 0.004), which is not significantly different from zero (p=0.43). Next, it was tested whether administration of a single bolus injection of INCB3284 would affect hemodynamics and fluid requirements in a short-term model of HS, when compared with vehicle treated animals. As an additional control drug, the CCR5 antagonist Maraviroc was tested. All animals were indistinguishable at baseline. To achieve a MAP of 30 mmHg during the HS period, vehicle-treated animals were hemorrhaged 49.7±1.9% total blood volume (TBV). Fluid requirements to maintain target blood pressures averaged 52.1±5.5 mL/kg after vehicle treatment (FIGS. 2A and 2B). All vehicle-treated animals survived the resuscitation period. To assess whether cognate agonists of CCR2, CCR3 and CCR5 are systemically released after hemorrhagic shock and fluid resuscitation in rats under experimental conditions, plasma levels of CCL2 (CCR2/3/5 agonist), CCL5 (CCR1/3/5 agonist), CCL7 (CCR1/2/3 agonist), CCL11 (CCR2/3/5 agonist) and CXCL10 (CCR3 and CXCR3 agonist) were measured in vehicle treated animals (Alexander S P, et al., Br J Pharmacol 2017; 174 Suppl 1:S17-S129). While CCL7 was not detectable, systemic concentrations of CCL2, CCL5 and CCL11 per mg of total protein significantly increased during fluid resuscitation from HS. FIGS. 2C-2F show the effects of the chemokine receptor antagonists when administered at the beginning of fluid resuscitation. The hemorrhage volumes to produce the target MAP during the shock period were comparable among all groups (p>0.05 vs. vehicle treated animals). All animals could be resuscitated with crystalloids to the target blood pressures and survived the observation period. While administration of 1.1 μmol/kg of INCB3284 did not affect fluid requirements to maintain hemodynamics (50±1 mL/kg, p>0.05 vs. vehicle), 5.5 μmol/kg INCB3284 reduced fluid requirements by 58±11% (21.7±6 mL/kg, p<0.05 vs. vehicle and 1.1 μmol/kg INCB3284) and increased MAP at various time points during the observation period (FIGS. 2C and 2D). Administration of 5.5 μmol/kg of Maraviroc did not affect fluid requirements, when compared with vehicle treated animals or with animals treated with 1.1 mmol/kg of INCB3284 (FIGS. 2E and 2F). To assess whether these fluid-sparing effects of INCB3284 could have therapeutic potential, it was tested whether a single dose of 5 μmol/kg of drug would have beneficial effects over a clinically more relevant observation period of 300 min (FIGS. 3A-3G). Hemorrhage volumes to achieve a MAP of 30 mmHg were comparable among vehicle-treated and INCB3284-treated animals (FIG. 3A). Vehicle-treated animals required on average 163±22 mL/kg of resuscitation fluid (FIGS. 3B and 3C). With INCB8284 treatment, average fluid requirements were reduced by 62±6% (61.9±10 mL/kg, p<0.05). FIGS. 3D and 3E show the fluid requirements for all individual animals treated with vehicle and INCB3284, respectively. Vehicle-treated animals that did not survive the observation period showed a steep increase in fluid requirements preceding cardiovascular collapse (FIG. 3D; marked with arrows). This is the turning point of the fluid requirements as the beginning of hemodynamic decompensation. Eight of the ten vehicle-treated animals reached the beginning of hemodynamic decompensation. The median time to the beginning of hemodynamic decompensation was 233.5 min with vehicle treatment (FIG. 3F). Three vehicle-treated animals that reached this turning point after t=210 min survived until t=300 min. None of the INCB3284-treated animals demonstrated the beginning of hemodynamic decompensation (FIGS. 3E and 3F; p<0.01 vs. vehicle treated animals). Hematocrit values were indistinguishable between groups. Lactate concentrations increased to approximately 10 mmol/L at the end of the HS period (p>0.05 between groups) and partially normalized in vehicle and INCB3284-treated animals. Partial pressures of O₂/CO₂ in arterial blood and creatinine concentrations were indistinguishable between groups. Mortality was 50% in vehicle-treated animals and 0% in INCB3284-treated animals (p<0.05, FIG. 3G). Median survival time was 295 min with vehicle treatment and >300 min with INCB3284 treatment (p<0.05 vs. vehicle). In animals that survived until t=300 min, lung wet weight/dry weight ratios were significantly reduced with INCB3284 treatment (FIG. 4A). Although differences between vehicle and INCB3284 treated animals in small bowel and colon wet weight/dry weight ratios did not reach statistical significance individually (FIGS. 4B and 4C), wet weight/dry weight ratios of all tissues combined were significantly reduced with INCB3284 treatment (FIG. 4D).

### Example 2

INCB3284 and SB328437 were purchased from Tocris, Bio-Techne Corporation (Minneapolis, MN, USA).

All procedures were performed in accordance with the National Institutes of Health Guidelines for Use of Laboratory Animals and were approved by the Institutional Animal Care and Use Committee of the University of South Florida (IS00008139). The IACUC specifically reviewed and approved the anticipated mortality in the study design. Male Sprague-Dawley rats (300-405 g) were purchased from Envigo (Indianapolis, IN, USA).

The hemorrhagic shock models were performed as described previously, with slight modifications (DeSantis A J, et al., The Chemokine (C-C Motif) Receptor 2 Antagonist INCB3284 Reduces Fluid Requirements and Protects From Hemodynamic Decompensation During Resuscitation From Hemorrhagic Shock. Crit Care Explor 2022;4(5):e0701; Bach H H, et al., Chemokine (C-X-C motif) receptor 4 and atypical chemokine receptor 3 regulate vascular alpha(1)-adrenergic receptor function. Mol Med 2014; 20:435-47; Nassoiy S P, et al., Effects of the Kv7 voltage activated potassium channel inhibitor linopirdine in rat models of hemorrhagic shock. Clin Exp Pharmacol Physiol 2018. doi: 10.1111/1440-1681.12958. PubMed PMID: 29702725; Bach H H, et al., Proteasome inhibition prolongs survival during lethal hemorrhagic shock in rats. J Trauma Acute Care Surg 2013; 74(2):499-507). In brief, anesthesia induction was performed with the animal and isoflurane-soaked gauze placed in a bell jar. After anesthesia induction, the animals were transferred to the operative field and anesthesia was maintained with 2.7% isoflurane administered via nose-cone inhalation with the SomnoSuite small animal anesthesia system (Kent Scientific Corporation, Torrington, CT, USA). At this dose of isoflurane rats did not respond to noxious stimuli but maintained spontaneous respiration. Using a direct cut-down technique, the left femoral artery was isolated and cannulated with a 24-gauge peripheral intravenous catheter to allow for blood withdrawal, hemodynamic monitoring, drug administration, and fluid resuscitation. After catheter placement, isoflurane was decreased to 1.5%. Hemodynamics were continuously monitored with the Surgivet invasive blood pressure monitor (Med-Electronics, Beltsville, MD, USA). Blood pressures were recorded at 1-5 min intervals during the hemorrhage and resuscitation periods. Four subsequent series of experiments were performed. All experiments in each series were performed in alternating order. In all experiments animals were continuously monitored and remained under general anesthesia until euthanasia or death, as defined by asystole or loss of pulse pressure.

Series 1: To assess the effects of SB328437 treatment after hemorrhagic shock and fluid resuscitation, and to be able to compare findings with the previous study on the effects of CCR2 and CCR5 inhibition, rats were hemorrhaged to a mean arterial blood pressure (MAP) of less than or equal to 30 mmHg for a period of 30 minutes. At the end of the hemorrhagic shock period (t=30 min), animals were injected with either vehicle (1 mL lactated Ringer's solution (LR), n=5), 0.25 μmol/kg SB328437 (n=3) or 1.1 μmol/kg SB328437 (n=3) in 1 mL LR, followed by fluid resuscitation with 1 mL bolus injections of LR to maintain a systolic blood pressure of 90 mm Hg or an MAP of 60 mm Hg until t=90 min, as described. To avoid fluid overload, bolus injections of LR were limited to 1 mL/min. At t=90 min, surviving animals were euthanized (5% isoflurane inhalation, bilateral pneumothorax).

Series 2: Rats were hemorrhaged to a MAP of less than or equal to 30 mmHg for a period of 60 min, followed by fluid resuscitation until t=300 min as in Series 1. At the end of the hemorrhagic shock period (t=60 min), animals were injected with 1 mL of normal saline (NS) (n=6), 5 μmol/kg (n=6) INCB3284 in 1 mL NS or 1.1 μmol/kg SB 328437 (n=6) in 1 mL NS at t=60 min. At t=0 min, 60 min, 120 min, 180 min, 240 min and 300 min, blood samples (0.3 ml) were obtained and used for arterial blood gas analyses and measurements of routine laboratory parameters. At t=300 min, surviving animals were euthanized (5% isoflurane inhalation, bilateral pneumothorax).

Series 3: Rats were hemorrhaged to a MAP of less than or equal to 30 mmHg for a period of 60 min, followed by fluid resuscitation until t=300 min as in Series 2. At the end of the hemorrhagic shock period (t=60 min) and at t=200 min, animals were injected with 1 mL of NS (n=7) or with 5 μmol/kg INCB3284 in 1 mL NS (n=7). At t=0 min, 60 min, 120 min, 180 min, 240 min and 300 min, blood samples (0.3 ml) were obtained and used for arterial blood gas analyses and measurements of routine laboratory parameters. At t=300 min, surviving animals were euthanized (5% isoflurane inhalation, bilateral pneumothorax).

Series 4: The purpose of these experiments was to determine whether treatment with the chemokine receptor antagonists increases shock tolerance, blood pressure and survival time without additional fluid resuscitation. As such, death is an intentional endpoint. Earlier endpoints are unable to answer these questions and alternatives are not available. Rats were hemorrhaged 40% total blood volume (TBV) within 10 min. At t=15 min, animals were injected with vehicle (1 mL NS, n=3), 5 μmol/kg INCB3284 in 1 mL NS (n=5) or 1.1 mol/kg SB 328437 in 1 mL NS (n=5). Animals were then hemorrhaged 2% TBV every 5 minutes until death.

Arterial blood gases and routine laboratory parameters. Arterial blood gases, electrolytes, creatinine, lactate, hematocrit and hemoglobin were analyzed using the Element point of care veterinary blood gas, electrolyte and critical care analyzer (Cuattro Veterinary USA, Loveland, CO, USA).

Data analyses and statistics. Data are presented as mean±standard error (SE). Data were analyzed 134 by 2-way analysis of variance (ANOVA) with Dunnett's multiple comparisons tests. Survival curves were analyzed using the log-rank test. Proportions were compared with the Fisher's exact test. All data analyses were calculated with the GraphPad Prism program (GraphPad Software Version 9.3.1). A two-tailed p<0.05 was considered significant.

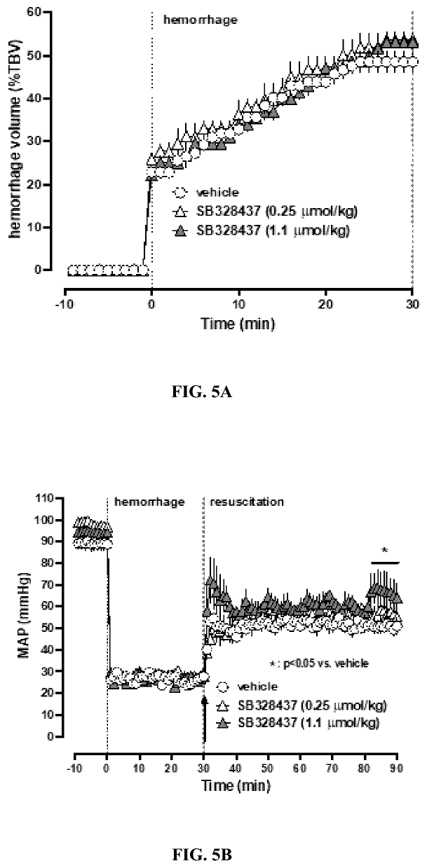

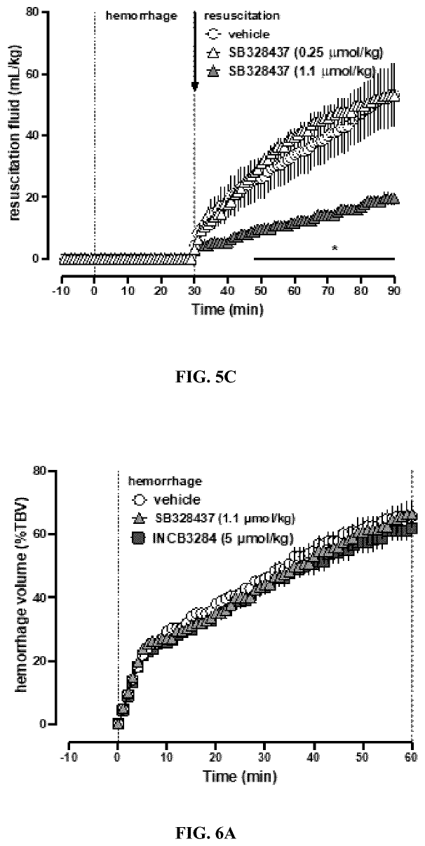

Series 1: single dose SB328437 treatment reduces fluid requirements within the first hour after a 30 min hemorrhagic shock period. To assess whether blockade of CCR3 affects hemodynamics and fluid requirements after hemorrhagic shock, the same animal model was employed that was previously utilized to characterize effects of the CCR2 and CCR5 antagonists INCB3284 and Maraviroc, respectively. Due to the limited solubility of SB328437, the highest dose of SB328437 that could be administered was 1.1 mol/kg. There were no differences in any physiological parameters among groups at baseline. The hemorrhage volumes to achieve the target MAP during the shock period were comparable among the groups (FIG. 5A). All animals could be resuscitated to the target MAP and survived the observation period (FIG. 5B). While MAP was indistinguishable between vehicle treated animals and animals treated with 0.25 μmol/kg SB328437, MAP at the end of the resuscitation period (t=80-90 min) was higher in animals treated with 1.1 mol/kg SB328437 (FIG. 5B). Fluid requirements to achieve the MAP target during the resuscitation period were comparable in vehicle-treated animals and in animals treated with 0.25 μmol/kg of SB328437 (FIG. 5C). As compared with vehicle-treated animals, fluid requirements 156 were reduced by 63±4% in animals treated with 1.1 μmol/kg SB328437 (fluid requirements (mean±SE): vehicle—53±10 mL/kg; 0.25 μmol/kg SB328437—53±7 mL/kg; 1.1 μmol/kg SB328437—19.5±2 mL/kg, p<0.05 vs. vehicle, FIG. 5C). These observations suggest that blockade of CCR3 with SB328437 dose dependently reduces fluid requirements in short term resuscitation experiments. The fluid sparing effects of 1.1 mol/kg SB328437 are comparable with the effects of 5 μmol/kg INCB3284 that were observed in the same model.

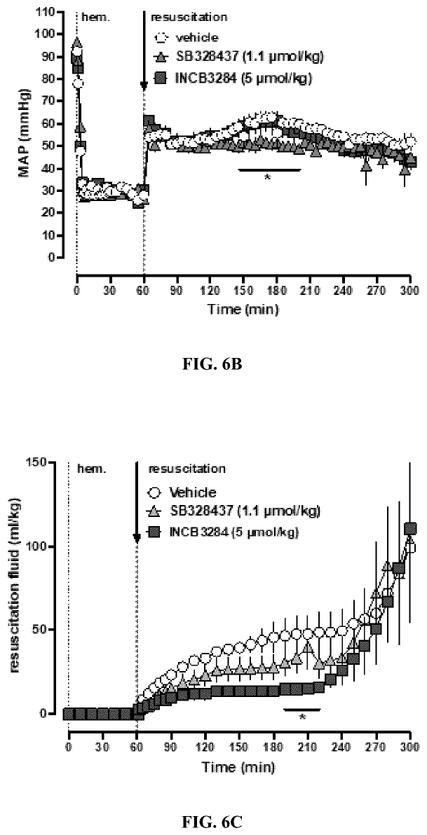

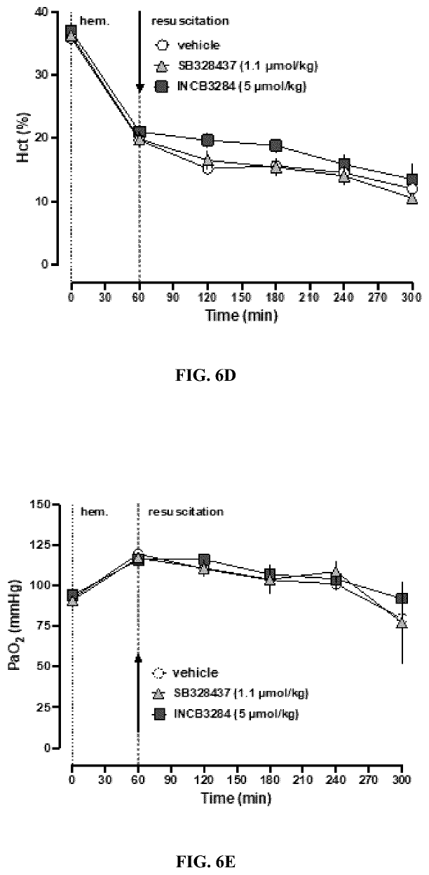

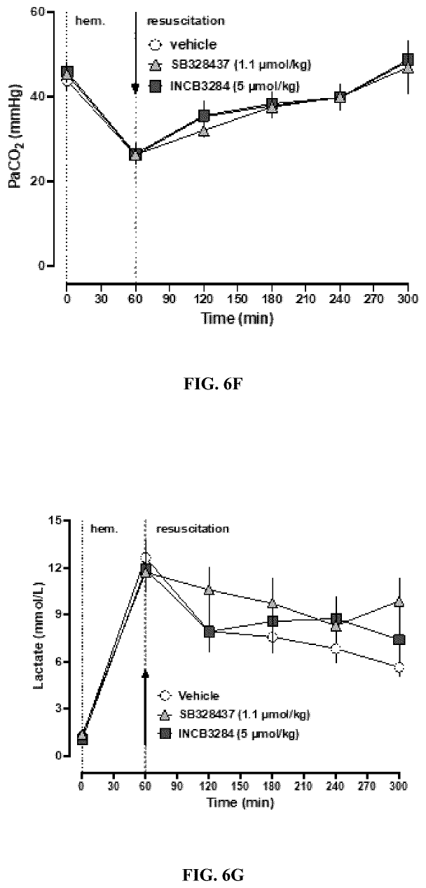

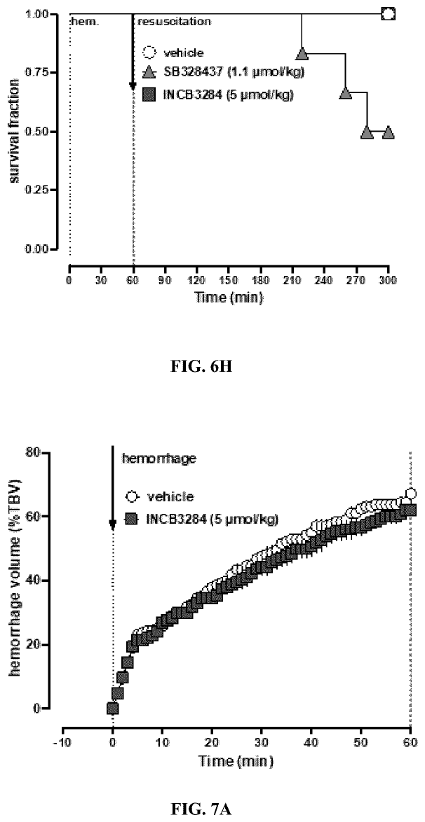

Series 2: single dose INCB3284 treatment, but not SB328437 treatment, transiently reduces fluid requirements after a 60 min hemorrhagic shock period. To assess therapeutic efficacy of SB328437 and INCB3284 in a more severe model of hemorrhagic shock and during clinically more relevant periods of fluid resuscitation, animals were treated with vehicle, 1.1 μmol/kg SB328437 or 5 μmol/kg INCB3284 178 after 60 min of hemorrhagic shock and performed fluid resuscitation until t=300 min. As in series 1, there were no differences among groups at baseline and the hemorrhage volumes to achieve MAP of <30 mmHg were indistinguishable (FIG. 6A). When compared with vehicle treatment, treatment with INCB3284 reduced fluid resuscitation requirements to achieve the target MAP by 65% until t=220 min (p<0.05 vs. vehicle, FIGS. 6B and 6C). With SB328437 treatment, however, MAP during fluid resuscitation was lower between t=140-200 min and fluid requirements could not be significantly reduced (FIGS. 6B and 10C). After t=220 min, all animals developed a steep increase in fluid requirements, suggesting hemodynamic decompensation. Hematocrit values (FIG. 6D), blood gases (FIGS. 6E and 6F) and lactate concentrations (FIG. 6G) were comparable among the groups. However, only 3 of 6 animals that were treated with SB328437 survived the resuscitation period, whereas all vehicle and INCB3284-treated animals survived (FIG. 6F). Median survival time with SB328437 treatment was 290 min and undefined with vehicle and INCB3284 treatment (p<0.05 vs. SB328437-treatment). It should be noted, however, that the number of animals in each group was small and survival proportions at t=300 min were not significantly different between SB328497 and vehicle treated animals (p=0.18). Because hematocrit values, blood gases and lactate levels were comparable among groups during the entire observation period and fluid requirements were indistinguishable after t=220 min, it cannot be excluded that some animals after vehicle or INCB3284 treatment would have died shortly after the end of the resuscitation period and that the median survival time is close to 300 min in all animals. Irrespective of a potential survival disadvantage, however, SB328437 treatment after 60 min of hemorrhagic shock lost the fluid sparing effect that were observed in series 1 after 30 min of hemorrhagic shock. In contrast, INCB3284 treatment transiently reduced fluid requirements until t=220 min after 60 min of hemorrhagic shock in the present study, suggesting that the therapeutic potential of INCB3284 to reduce fluid resuscitation requirements is higher than of SB328437.

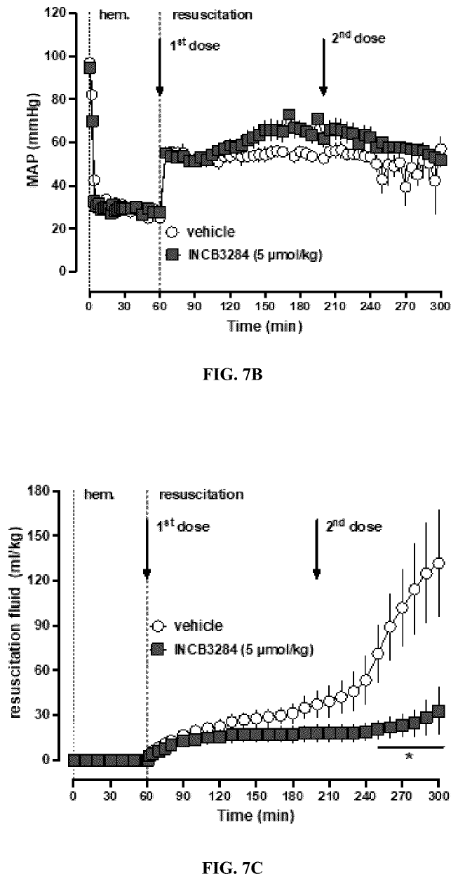

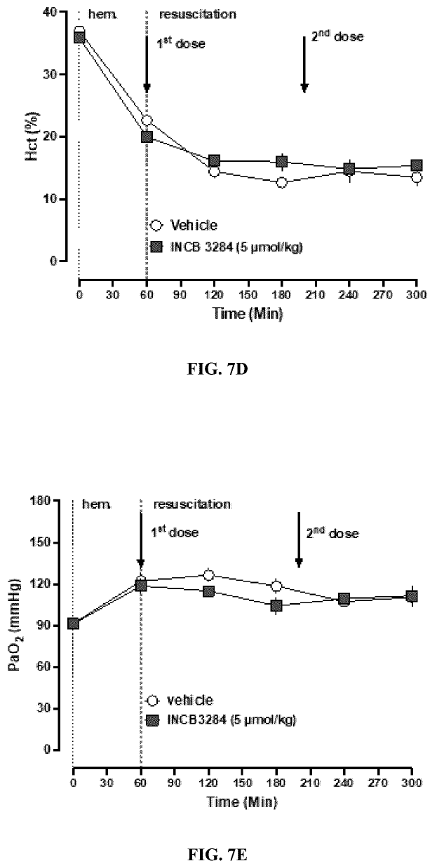

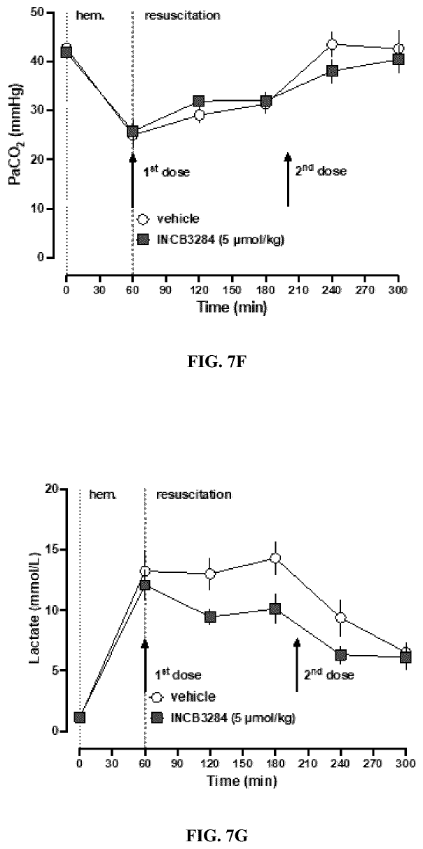

Series 3: redosing of INCB3284 reduces fluid requirements and prevents hemodynamic decompensation after a 60 min hemorrhagic shock period. The half-live of INCB3284 has been reported to be 168 min in rats (Xue C B, et al., Discovery of INCB3284, a Potent, Selective, and Orally Bioavailable hCCR2 Antagonist. ACS Med Chem Lett 2011; 2(6):450-4). In combination with the observations that 5 μmol/kg INCB3284 reduced fluid requirements after hemorrhagic shock, whereas a dose of 1.1 mol/kg INCB3284 was ineffective, it appeared possible that systemic concentrations of INCB3284 after t=200-220 min declined below the threshold of therapeutic efficacy. To test whether redosing of INCB3284 improves therapeutic efficacy, the same model as in series 2 was utilized and administered vehicle or INCB3284 at the beginning of fluid resuscitation (t=60 min) and at t=200 min. Similar to series 1 and 2, there were no differences among groups at baseline and the hemorrhage volumes to achieve MAP of <30 mmHg were indistinguishable (FIG. 7A). As expected, based on the findings in series 2, fluid requirements to maintain target MAP during resuscitation increased rapidly after t=220 min and reached 131±35 mL/kg at t=300 min after vehicle treatment (FIGS. 7B and 7C). With double dosing of INCB3284, the steep increase in fluid requirements was prevented and total fluid requirements were reduced by 75% (33±16 mL/kg, p<0.05 vs. vehicle treatment), when compared with vehicle treated animals (FIGS. 7B and 7C). As in series 2, hematocrit values, blood gases and lactate concentrations were not significantly different among groups (FIGS. 7D-7G). Unlike in series 2, mortality was 71% and median survival time 297 min after vehicle treatment (FIG. 7H). With double dosing of INCB3284, however, mortality was reduced to zero (p<0.05 vs. vehicle treatment) and median survival time undefined (p<0.05 vs. vehicle treatment, FIG. 7H).

Given that fluid requirements and other physiological parameters of vehicle treated animals were comparable in series 2 and 3, these observations suggest that the lack of mortality after vehicle treatment in series 2 was a chance observation caused by a small sample size, which argues against a survival disadvantage with single dose SB328437 treatment. The combined mortality in vehicle treated animals from series 2 and 3 (n=13 animals) was 38.5% and median survival time remains undefined, which is not significantly different from zero mortality that was observed with double dosing of INCB3284. This indicates that larger cohort sizes are required to clarify whether double dosing of INCB3284 provides a relevant survival benefit. Nevertheless, these finding that double dosing of INCB3284 prevented the steep increase in fluid requirements that was detectable in all vehicle treated animals in series 2 and 3 and in animals after single dose treatment with INCB3284 in series 2, demonstrates that dosing of INCB3284 can be optimized to increase its fluid sparing effects during resuscitation from hemorrhagic shock and suggests that treatment of animals with an optimized dosing regimen of INCB3284 also prevents, or at least delays, hemodynamic decompensation in a more severe model of hemorrhagic shock.

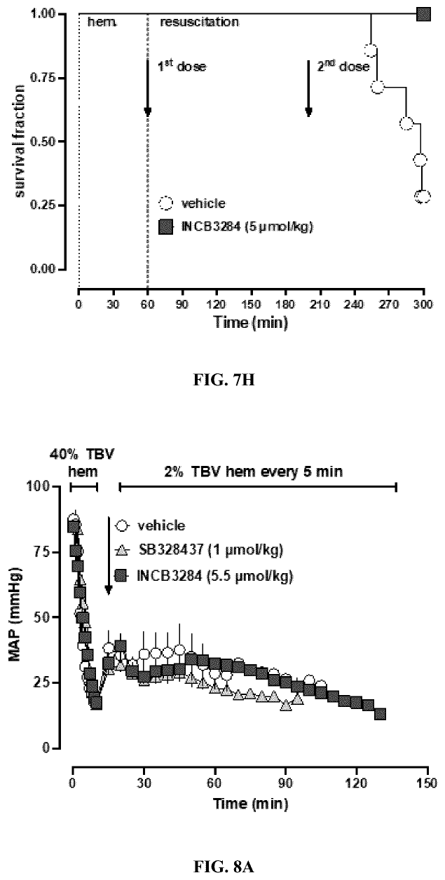

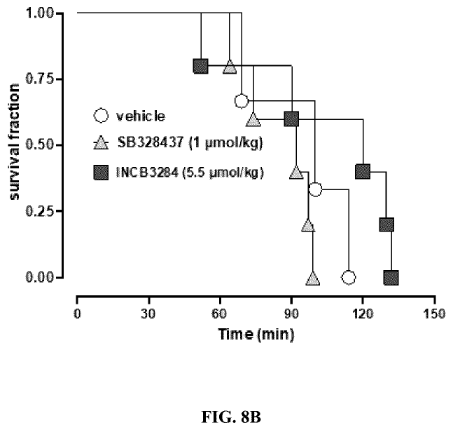

Series 4: treatment with INCB3284 and SB328437 does not increase shock tolerance. To assess whether INCB3284 or SB328437 may increase tolerance to severe hemorrhagic shock, a model designed to mimic continuous bleeding in the absence of fluid resuscitation was used. As shown in FIG. 8A, when animals were treated with vehicle, 5 mol/kg INCB3284 or 1 μmol/kg SB328437 after 40% TBV hemorrhage, MAP was indistinguishable among groups during subsequent 2% TBV hemorrhages in 5 min intervals. Median survival times were not significantly different between groups (median 268 survival time: vehicle treatment—72 min; SB328437—64 min; INCB328437—80 min, FIG. 8B).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

What is claimed is:

1. A method of fluid resuscitation of a patient in need thereof, comprising:
administering to the patient a resuscitation fluid and an effective amount of a C-C chemokine receptor (CCR) inhibitor.

2. The method of claim 1, wherein the patient has hemorrhagic shock.

3. The method of claim 1, wherein the patient is undergoing cardiovascular, abdominal, or transplant surgery.

4. The method of claim 1, wherein the patient is treated for ischemia, hypovolemic shock, myocardial infarction, or stroke.

5. The method of claim 1, wherein the patient is treated for trauma, burn, sepsis, or shock.

6. The method of claim 1, wherein the CCR inhibitor is a CCR2 and/or CCR3 antagonist.

7. The method of claim 1, wherein the CCR inhibitor is INCB3284 or SB328437.

8. The method of claim 1, wherein the CCR inhibitor is administered separately from the resuscitation fluid.

9. The method of claim 1, wherein the CCR inhibitor is administered in one or two doses.

10. The method of claim 1, wherein the CCR inhibitor is a component of the resuscitation fluid.

11. The method of claim 1, wherein the CCR inhibitor is administered at a dosage of from 1 to 10 mmol/kg of the patient.

12. The method of claim 1, wherein the resuscitation fluid is lactated Ringer's solution.

13. The method of claim 1, wherein the resuscitation fluid is administered in an amount that is less than three times the amount of fluid lost by the patient.
